(19) United States
(12) Patent Application Publication
(10) Pub. No.: US 2017/0240926 A1
(43) Pub. Date: Aug. 24, 2017

(54) METHODS FOR PRENYLATION OF PEPTIDES AND THEIR USE IN OVER-PRODUCTION OF FARNESENE AND GERANYLGERANYL TERPENES

(71) Applicant: University of North Texas, Denton, TX (US)

(72) Inventor: Richard McQualter, Denton, TX (US)

(21) Appl. No.: 15/436,402

(22) Filed: Feb. 17, 2017

Related U.S. Application Data
(60) Provisional application No. 62/297,615, filed on Feb. 19, 2016.

(51) Int. Cl. C12P 5/00; C07K 4/00

(52) U.S. Cl. CPC C12P 5/007; C07K 4/00

(57) ABSTRACT

Methods for concentrating biofuel precursors, including terpenes such as farnesyl and geranylgeranyl derivatives, are based on the prenylation of peptides in living organisms, such as plant or algae cells. Generally, an expression vector containing a gene encoding a small peptide with a preferred amino acid sequence is used to produce a transgenic organism. Expression of the gene in the cells produces a short peptide which is processed by the protein prenylation machinery of the cell. This results in a peptide-prenyl fusion in which a sesqui- or di-terpene molecule is attached to the peptide. Due to its small size and amphiphilic properties, this molecule forms micelles which allow the sesqui- or di-terpene to accumulate to high concentrations within the cell. The peptide-prenyl micelles are then extracted and purified for use preferably as a biofuel.

This application claims priority to U.S. Provisional Patent Application No. 62/297,615 entitled “Methods for Prenylation of Peptides and Their Use in Over-Production of Farnesene and Geranylgeranyl Terpenes,” filed on Feb. 19, 2016, the entire contents of which are hereby incorporated by reference.

The present invention used in part funds from the U.S. Department of Energy, Advanced Research Projects Agency, Grant No. DE-AR0000208. The United States Government has certain rights in the invention.

### BACKGROUND

This disclosure pertains to prenylation of peptides and the production of biofuels.

Biofuels offer renewable alternatives to petroleum-based fuels that reduce net greenhouse gas emissions to nearly zero. However, traditional biofuels production is limited not only by the small amount of solar energy that plants convert through photosynthesis into biological materials, but also by inefficient processes for converting these biological materials into fuels. Farm-ready, non-food crops are needed that produce fuels or fuel-like precursors at significantly lower costs with significantly higher productivity. To make biofuels cost-competitive with petroleum-based fuels, biofuels production costs must be cut in half.

Sesquiterpenes and diterpenes are fuel precursors that can be blended into diesel fuel. Synthesis of these compounds in large quantities in living organisms has so far proven problematic. This is because they can become toxic at high levels. Due to this toxicity, accumulation of large amounts of sesquiterpenes in living cells is difficult to achieve.

What is needed, therefore, is a mechanism to safely accumulate large amounts of biofuel precursors, such as sesquiterpenes and diterpenes, to facilitate the production of biofuels in a more cost-efficient manner.

### SUMMARY

The present disclosure relates generally to the over-production and accumulation of terpenes in living cells through the prenylation of peptides in those cells.

The present methods and uses relate to a mechanism for concentrating farnesyl and geranylgeranyl derivatives in living organisms, such as plant or algae cells, at high concentrations. Generally, an expression vector containing a gene encoding a small peptide with a preferred amino acid sequence is used to produce a transgenic organism, such as a plant or algae. Expression of the gene in the cells produces a short peptide which is processed by the protein prenylation machinery of the cell. This results in a peptide-prenyl fusion in which a sesqui- or di-terpene molecule is attached to the peptide. Due to its small size and amphiphilic properties, this molecule forms micelles which allow the sesqui- or di-terpene to accumulate to high concentrations within the cell. The peptide-prenyl micelles are then extracted and exposed to a reducing agent. This breaks the sulphur bond between the peptide and the sesqui- or di-terpene molecule, allowing purification of the terpene for use preferably as a biofuel.

Prenylation or isoprenylation is a post-translational modification process essential for the proper localization and function of many proteins. Some eukaryotic proteins, including those with cysteine residues close to the C-terminal regions, are biosynthetically modified with an isoprenoid lipid, such as the 15-carbon farnesyl group or the 20-carbon geranylgeranyl group (Zhang et al., 1996). Prenylation provides some proteins with a hydrophobic membrane anchor, and is important for their correct localization within the cell. FIG. 1 shows an illustrations of some mechanisms by which proteins are anchored to cell membranes through the use of an isoprenoid lipid such as farnesyl (C₁₅) or geranylgeranyl (C₂₀).

Protein farnesyltransferase (FTase) and protein geranylgeranyltransferase type I (GGTase-I) are members of the prenyltransferase family of sulfur alkyltransferases. These enzymes, both heterodimers of α and β subunits, use a zinc ion to catalyze the covalent attachment of a 15-carbon farnesyl group from farnesyl diphosphate (FPP) or a 20-carbon geranylgeranyl group from geranylgeranyl diphosphate (GGPP) to the thiol side chain of a cysteine residue near the C-terminus of a protein substrate. This is illustrated in FIG. 2. A protease removes the three terminal amino acids, leaving cysteine as the C-terminal amino acid. Methylation then converts the C-terminal residue from a negatively charged, hydrophilic group to an uncharged, hydrophobic group and increases membrane affinity approximately 10-fold.

Both FTase and GGTase-I are understood to recognize protein or peptide substrates containing certain C-terminal amino acid sequences as targets for prenylation. A short peptide containing the appropriate motif for FTase or GGTase recognition and activity will act as a means both for the creation of farnesyl and geranylgeranyl derivatives and their safe sequestering in micellular compartments such as those shown in FIG. 3.

Sequestering the farnesyl and geranylgeranyl derivatives into micellular components produces two unique advantages. First, it protects the cell from the toxicity of the terpenes. One way to detoxify a molecule is to attach it to a carrier molecule, thus changing its chemical properties. Second, formation of the micelles also increases demand by removing product from the end of the terpene biosynthesis pathway. Demand is responsible for the majority of flux control through biochemical pathways and supply rate drops when consumption is reduced. Hence, removing the end product (e.g. by polymerization, crystal formation, glycosylation, export to another compartment) changes the equilibrium of a pathway and increases flux. One way to increase flux to terpene synthesis therefore would be to remove it by addition of a carrier molecule via prenylation, resulting in the formation of micelles that isolate the terpenes from the ongoing biochemical processes.

### BRIEF DESCRIPTION OF THE DRAWINGS

FIG. 1 shows an illustration of mechanisms by which proteins are anchored to cell membranes with an isoprenoid lipid.

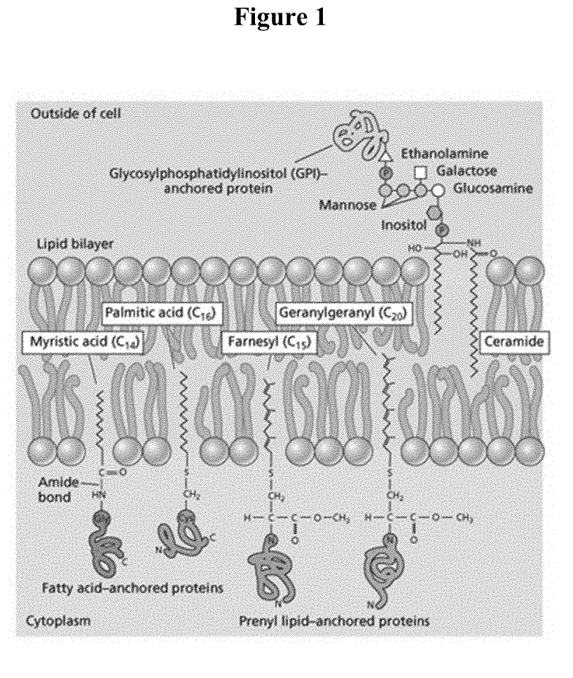

FIG. 2 shows an illustration of the enzymatic steps that are involved in the prenylation of proteins.

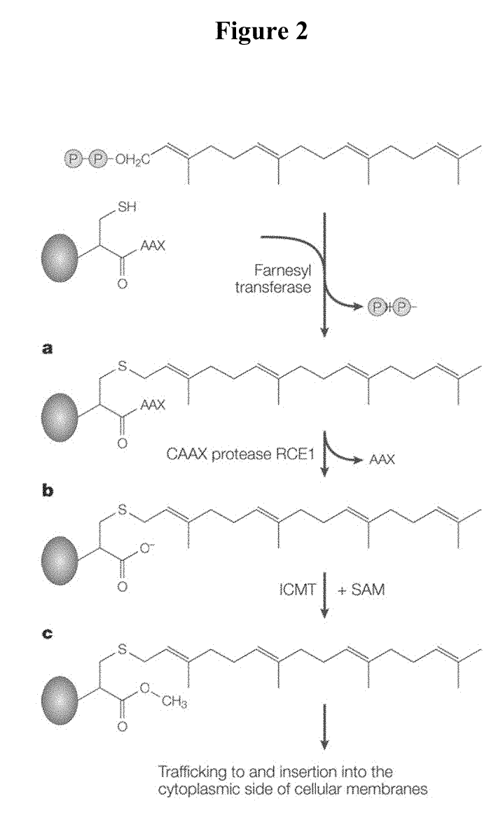

FIG. 3 shows a representation of a micelle formed from prenylated proteins.

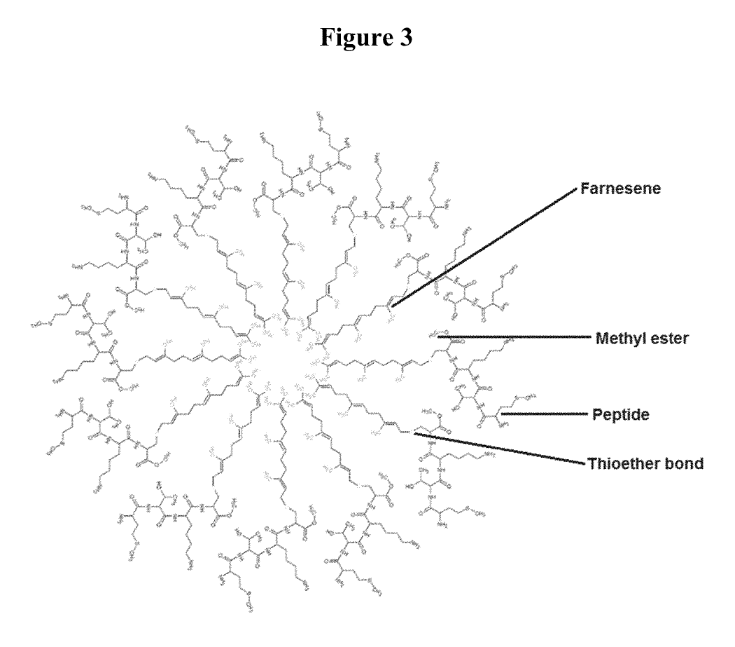

FIG. 4 shows (A) an example of a possible nucleotide coding sequence and amino acid sequence of a gene for a peptide targeted for prenylation and (B) an example of a possible plasmid map for a plant or algae expression vector containing a prenyl peptide gene driven by a plant or algae specific promoter.

### DETAILED DESCRIPTION OF PREFERRED EMBODIMENTS

Generally, the present disclosure relates to production of biofuels, particularly fuel precursors such as terpenes, in living cells through the prenylation of peptides.

In preferred embodiments, a peptide and its underlying nucleic acid sequence are designed to include an appropriate motif for protein farnesyltransferase (FTase) or protein geranylgeranyltransferase type I (GGTase-I) recognition and activity. It is understood that both FTase and GGTase-I recognize protein or peptide substrates containing a C-terminal “Ca1a2X” sequence in which a1 and a2 are aliphatic amino acids and X represents methionine (M), serine (S), glutamine (Q), alanine (A), or cysteine (C) for FTase and leucine (L) or glutamic acid (E) for GGTase. Table 1 below shows the kinetic and binding constants of farnesylated substrates from a peptide library that was screened to identify sequences that exhibited multiple turnover reactivity with FTase (Houghland et al., 2000). The peptides, including the last four amino acids and the N-terminal Thr and Lys residues (TKCxxx), were screened for farnesylation by FTase. The lysine residue upstream of Cxxx increases the solubility of the peptide, while the basic amino acid threonine enhances dual specificity for prenylation by both farnesyltransferase and geranylgeranyltransferase.

TABLE 1
(reproduced from Houghland et al. 2000)

| | | k_{cat}/Kₘ | k_{farn} | | |
|---|---|---|---|---|---|
| Peptide^{a} | Protein name(s) | (mM⁻¹ s⁻¹) | (s⁻¹) | K_{d} (nM) | Pred^{b} |
| CAVQ | Rab28 | 44 ± 5 | 11 ± 1 | 13 ± 3 | Yes |
| CSIM^{c} | Rho6 and Lamin A/C | 14 ± 1 | 9.7 ± 0.9 | 6 ± 2 | Yes |
| CLVM | Rho-related BTB domain | 6 ± 1 | 8.2 ± 0.8 | 6 ± 2 | Yes |
| | containing protein | | | | |
| CTKF | Neuronal membrane | 4.4 ± 0.4 | 0.24 ± 0.02 | 6 ± 1 | No |
| | glycoprotein M6-b | | | | |
| CRIS | Phosphatase 1 regulatory | 3.8 ± 0.4 | 2.5 ± 0.5 | 12 ± 2 | Yes |
| | subunit | | | | |
| CCIF | Cell division cycle 42 | 3.0 ± 0.3 | 0.59 ± 0.06^{d} | 270 ± 70 | Yes |
| | (GTP binding) | | | | |
| CKIS | Rab-40(A/B/C) | 2.8 ± 0.6 | 0.64 ± 0.06 | 4.0 ± 0.8 | No |
| CSIS | Hypothetical protein | 2.7 ± 0.3 | 3.8 ± 0.3 | 3.4 ± 0.8 | Yes |
| CYVM | Lamin B2 | 2.5 ± 0.6 | 1.1 ± 0.1 | 12 ± 4 | Yes |
| CLIT | RhoQ | 2.5 ± 0.3 | 0.74 ± 0.07 | 4.1 ± 0.8 | Yes |
| CNIM | Hypothetical protein | 2.1 ± 0.2 | 3.5 ± 0.3 | 5 ± 1 | No |
| CTLQ | Hypothetical protein | 1.9 ± 0.1 | 1.2 ± 0.1 | 7 ± 1 | Yes |
| CSVS | Phosphatidylinositol | 1.8 ± 0.2 | 0.97 ± 0.09 | 2.1 ± 0.9 | Yes |
| | polyphosphate 5- | | | | |
| | phosphatase | | | | |
| CSLM | Ras-like protein RRP22 | 1.8 ± 0.4 | 7.0 ± 0.7 | 8 ± 2 | Yes |
| CLLM | Phosphatase 1 regulatory | 1.7 ± 0.3 | 4.9 ± 0.5 | 4.7 ± 0.9 | Yes |
| | subunit | | | | |
| CTVF | Ras-related C3 botulinum | 1.3 ± 0.1 | 0.40 ± 0.04 | ND^{e} | Yes |
| | toxin substrate 3 | | | | |
| CSVQ | GTP binding protein Rab28 | 1.2 ± 0.4 | 4.4 ± 0.5 | ND^{e} | Yes |
| | (splice variant) | | | | |
| CGIM | Glutathione transferase | 1.1 ± 0.3 | 4.0 ± 0.4 | 3.6 ± 0.7 | Yes |
| CTLM | GTP-binding tumor | 1.1 ± 0.2 | ND^{e} | 5.2 ± 0.8 | Yes |
| | suppressor 1 | | | | |
| CGLY | Guanine nucleotide binding | 1.0 ± 0.1 | 0.13 ± 0.01 | ND^{e} | No |
| | protein G(O) alpha | | | | |
| | subunits 1 and 2 | | | | |
| CNLM | Rho-related GTP binding | 1.0 ± 0.3 | 3.1 ± 0.3 | ND^{e} | Yes |
| | protein RhoN | | | | |
| CYPD | Immunoglobulin J chain | 0.9 ± 0.1 | 0.56 ± 0.05 | 3.3 ± 0.9 | No |
| CVLQ | Hypothetical | 0.78 ± 0.03 | ND^{e} | ND^{e} | Yes |
| | protein/ubiquitin-specific | | | | |
| | processing protease 32 | | | | |
| CITQ | Stoned B-like factor SBLF | 0.71 ± 0.05 | 1.1 ± 0.1 | 3.6 ± 0.8 | Yes |
| CFLS | SCAMPER | 0.6 ± 0.1 | 0.46 ± 0.05 | 19 ± 6 | Yes |
| CNLQ | Tetraspanin 1/lens-derived | 0.67 ± 0.04 | 1.6 ± 0.2 | 8 ± 1 | No |
| | growth factor | | | | |
| CTMS | TRIP protein | 0.53 ± 0.03 | 0.91 ± 0.09 | 6 ± 2 | Yes |
| CQTA | DNA J homolog subfamily | 0.53 ± 0.02 | 0.23 ± 0.02 | ND^{e} | Yes |
| | A member 4 | | | | |
| CVLA | CAAX box protein 1 | 0.5 ± 0.1 | 0.51 ± 0.05 | 4.6 ± 0.9 | Yes |
| | (cerebral protein-5) | | | | |
| CGLF | Guanine nucleotide binding | 0.45 ± 0.06 | 0.051 ± 0.005 | ND^{e} | No |
| | protein alpha-1 subunit | | | | |
| CIVA | LIM-only protein 6 and | 0.44 ± 0.07 | 4.7 ± 0.5 | 5 ± 2 | Yes |
| | LIM domain only 6 | | | | |
| CSFM | Ras-related protein Rab-37 | 0.39 ± 0.04 | 0.44 ± 0.04 | 10 ± 3 | No |
| CTLA | Hypothetical protein | 0.36 ± 0.04 | 0.015 ± 0.002 | 4.4 ± 0.7 | No |
| CCIL | Rod cGMP-specific 3′,5′ | 0.32 ± 0.07 | 0.13 ± 0.02^{d} | 300 ± 40 | Yes |
| | cyclic phosphodiesterase | | | | |
| | beta subunit | | | | |
| CVQM | Cystatin M precursor | 0.27 ± 0.03 | 1.7 ± 0.2 | ND^{e} | No |
| CVVT | RhoD | 0.27 ± 0.02 | 0.33 ± 0.03 | 7 ± 3 | No |
| CSLG | Ras-related protein Rab-13 | 0.24 ± 0.03 | 0.059 ± 0.006 | 3.4 ± 0.7 | No |
| CSLC | LIM-only protein 6 | 0.24 ± 0.05 | 4.7 ± 0.5 | 5 ± 2 | Yes |
| CLTS | Hypothetical protein | 0.22 ± 0.03 | 0.35 ± 0.03 | 5 ± 1 | No |
| CKIF | Rho-related GPT binding | 0.17 ± 0.04 | 0.74 ± 0.07 | 2.5 ± 0.5 | Yes |
| | protein RhoH | | | | |
| CNLT | Hypothetical protein | 0.17 ± 0.03 | 0.18 ± 0.02 | 3.7 ± 0.7 | No |
| CGIA | Hypothetical protein | 0.16 ± 0.05 | 0.75 ± 0.07 | ND^{e} | Yes |
| CVLH | Hypothetical protein | 0.15 ± 0.03 | 0.32 ± 0.03 | 14 ± 3 | No |
| CLML | Cone cGMP-specific 3′,5′ | 0.15 ± 0.02 | 0.018 ± 0.002 | ND^{e} | Yes |
| | cyclic phosphodiesterase | | | | |
| | alpha subunit | | | | |
| CTII | 2′,3′-Cyclic nucleotide 3′- | 0.092 ± 0.002 | 0.25 ± 0.03 | 4.2 ± 0.9 | Yes |
| | phosphodiesterase | | | | |

Assays include 10 μM FPP, 0.4-10 μM dansylated peptide, 25-300 nM FTase, 50 mM HEPPSO buffer, pH 7.8, 5 mM TCEP, 5 mM MgCl2, and 10 μM ZnCl (at 25° C.).
^{a}Peptides are of the form dansyl-TKCxxx, where x represents any amino acid.
^{b}Predicted to be farnesylated based on Refs. 9 and 10.
^{c}Sequences identified as farnesylated before or during this study.
^{d}The peptide concentration in this assay was increased to 50 μM.
^{e}ND, not determined.

In preferred embodiments, a peptide sequence containing a sequence in the form of CaaX, where X may represent the amino acids methionine, serine, glutamine, alanine, cysteine, leucine or glutamate (e.g. CAVQ, see Table 1) is selected as the peptide showing favorable kinetic properties as a substrate for FTase or GGTase. A lysine residue upstream of Cxxx is preferably included to increase the solubility of the peptide, while a basic amino acid such as threonine is preferably included to enhance dual specificity for prenylation by both farnesyltransferase and geranylgeranyltransferase. A methionine is preferably included as a requisite for translation in a eukaryotic system and an aspartate residue is preferably added between the M and T residues to increase hydrophilicity of the peptide and to allow the inclusion of the Kozak consensus sequence (CCATGG) in the DNA construct at the site of translation initiation. The Kozak consensus sequence plays a major role in the initiation of the translation process. The overall peptide preferably has the form of MDTKCaaX but may contain additional amino acids between the N-terminal methionine and the threonine residue.

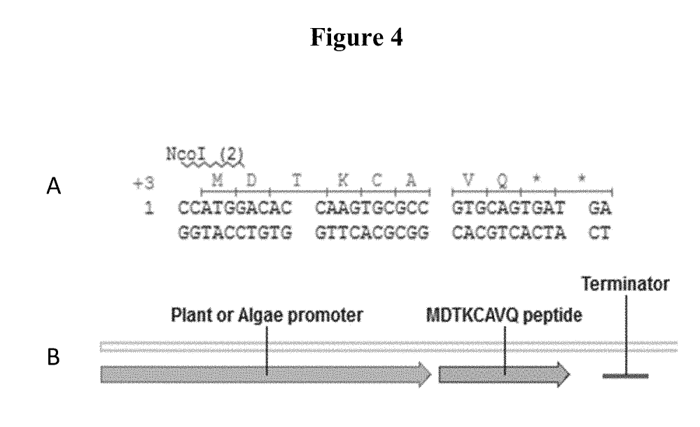

In additional preferred embodiments, a DNA sequence coding for MDTKCaaX and preferably codon optimized for the intended host organism is synthesized (see FIG. 4a) and cloned into a plant expression vector preferably driven by a constitutive promoter such as the CAMV 35S promoter or the maize ubiquitin promoter (see FIG. 4b) or an algae expression vector driven by an algal promoter such as Hsp70A-Rbc S2 chimeric constitutive promoter. The expression vector is introduced into plant or algae cells preferably using Agrobacterium mediated transfer (plants), microprojectile bombardment (plants or algae), electroporation (algae) or thee glass bead method (algae) along with a suitable selectable marker gene (e.g. Neomycin phosphotransferase, Zeocin). Transgenic plants/algae are generated using standard methods. Plants/algae are analyzed for increased terpene content. The thioether bond linking the peptide to the prenyl group can be cleaved by hydrogenolysis using a reducing agent such as Raney nickel.

Preferred embodiments include methods for concentrating farnesyl and geranylgeranyl derivatives in the leaves of plants or in algae cells at high concentrations. An additional preferred embodiment is a plant/algae expression vector containing a gene encoding a small peptide with amino acid sequence of the form MX(n)TKCaaX that is used to produce a transgenic plant/algae. Expression of the gene in plant/algae cells produces a short peptide which is processed by the protein prenylation machinery of the cell. This would result in a peptide-prenyl fusion in the form of MX(n)TKC-prenyl. Due to its small size and amphiphilic properties, this molecule forms micelles which allow the sesqui- or di-terpene to accumulate to high concentrations. The peptide-prenyl micelles are then extracted and exposed to a reducing agent such as Raney nickel. This breaks the sulphur bond between the peptide and sesqui- or di-terpene molecule, allowing purification of terpene for use as a biofuel or other desirable material.

Preferred embodiments of the peptide are in the form MX(n)TKCaaX to be recognized by the plant/algae's prenylation machinery for prenylation. A lysine residue upstream of CaaX is included to increase the solubility of the peptide, while a basic amino acid such as threonine is included to enhance dual specificity for prenylation by both farnesyltransferase and geranylgeranyltransferase. Additional amino acid residues may be added to increase the hydrophilicity of the peptide portion of the molecule. A methionine is included as a requisite for translation in a eukaryotic system.

In additional embodiments, incorporation of an amino acid tag such as a polyhistidine tag to the N-terminal sequence of the short peptide (e.g. MHHHHHHTKCaaX) enables simplified purification of the prenylated peptide by nickel column. Polyhistidine-tags are often used for affinity purification of polyhistidine-tagged recombinant proteins. Other affinity tags such as the FLAG sequence (N-AspTyrLysAspAspAspAsp-Lys-C) might also be used for capturing the terpene-peptide conjugate.

Preferred embodiments of the methods and uses described herein include a method for concentrating and collecting biofuel precursors, namely, terpenes, from living cells. In a first step, the living cell is modified so that it expresses a peptide that is targeted for prenylation by the cell. In preferred embodiments, this modification is by transforming the cell to include an expression vector for expressing a peptide that is targeted for prenylation. In preferred embodiments, the living cell is a plant or algae and the expression vector is driven by a promoter, which may be a constitutive promoter, specific for the plant or algae. The cell may, however, be any suitable cell such as bacteria, yeast, or any other living cell that can be modified to express the peptides. The cell then expresses the peptide, the peptides are prenylated with a terpene molecule, such as farnesene, and the prenylated peptides accumulate into micelles within the living cells. The micelles are then extracted from the cells and exposed to a reducing agent to break the bond between the terpene and the peptide. The terpene molecules are then purified for use as biofuel precursors, or for other suitable uses.

In additional preferred embodiments, the expression vector comprises a nucleotide sequence coding for an amino acid sequence of CaaX, where a is any aliphatic amino acid and X is methionine (M), serine (S), glutamine (Q), alanine (A), cysteine (C), leucine (L) or glutamate (E). In further preferred embodiments, the expression vector comprises a nucleotide sequence coding for an amino acid sequence of TKCaaX, where T represents threonine, K represents lysine, and CaaX has the definition provided above. In further preferred embodiments, the expression vector comprises a nucleotide sequence coding for an amino acid sequence of MY(n)TKCaaX, where M represents methionine and Y is one or more additional amino acids. The variable n is an integer representing the number of amino acids included in Y. When n is 1, Y is aspartate or glutamate. When n is 2 or more, at least one amino acid in Y, immediately adjacent to the methionine, is aspartate or glutamate. In additional embodiments, the expression vector comprises a nucleotide sequence that includes the Kozak consensus sequence (CCATGG).

### REFERENCES CITED

The following documents and publications are hereby incorporated by reference.

### Non-Patent Publications

Zhang, Fang L. and Patrick J. Casey, Protein Prenylation: Molecular Mechanisms and Functional Consequences, Annual Review of Biochemistry, Vol. 65: 241-269 (1996).
Hougland, J. L., Hicks, K. A., Hartman, H. L., Kelly, R. A., Watt, T. J., & Fierke, C. A., Journal of Molecular Biology, 395(1), 176-190 (2010).

What is claimed is:

1. A method for concentrating and collecting terpenes using living cells, comprising:
modifying the living cells to cause the living cells to express peptides that are targeted for prenylation, whereby the living cells express the peptides, the peptides are prenylated with terpene molecules by the living cells to produce prenylated peptides, and the prenylated peptides accumulate into micelles inside the living cells;
extracting the micelles from the living cells;
releasing the terpene molecules from the prenylated peptides; and
collecting the terpene molecules.

2. The method of claim 1, wherein the step of modifying the living cells comprises transforming the living cells with an expression vector that causes the living cells to express the peptides that are targeted for prenylation.

3. The method of claim 2, wherein the expression vector comprises a nucleotide sequence that codes for an amino acid sequence of CaaX, wherein C is cysteine, a is any aliphatic amino acid and X is methionine (M), serine (S), glutamine (Q), alanine (A), cysteine (C), leucine (L) or glutamate (E).

4. The method of claim 2, wherein the expression vector comprises a nucleotide sequence that codes for an amino acid sequence of TKCaaX, wherein C is cysteine, a is any aliphatic amino acid, X is methionine (M), serine (S), glutamine (Q), alanine (A), cysteine (C), leucine (L) or glutamate (E), T is threonine, and K is lysine.

5. The method of claim 2, wherein the expression vector comprises a nucleotide sequence that codes for an amino acid sequence of MY(n)TKCaaX, wherein C is cysteine, a is any aliphatic amino acid, X is methionine (M), serine (S), glutamine (Q), alanine (A), cysteine (C), leucine (L) or glutamate (E), T is threonine, and K is lysine, M is methionine, Y is one or more additional amino acids, and n is an integer representing a total number of the amino acids included in Y.

6. The method of claim 5, wherein n is 1 and Y is aspartate (D) or glutamate (E).

7. The method of claim 5, wherein n is more than 1 and Y comprises at least one amino acid that is aspartate (D) or glutamate (E) immediately adjacent to the methionine (M).

8. The method of claim 2, wherein the expression vector comprises a nucleotide sequence that comprises the Kozak consensus sequence CCATGG.

9. The method of claim 2, wherein the expression vector comprises a nucleotide sequence that comprises a promoter specific for the living cells.

10. The method of claim 9, wherein the promoter is constitutive.

11. The method of claim 1, wherein the living cells are plant or algae cells.

12. The method of claim 1, wherein the step of releasing the terpene molecules comprises exposing the micelles to a reducing agent.

13. The method of claim 1, wherein the step of collecting the terpene molecules further comprises purifying the terpene molecules.

14. The method of claim 1, further comprising the step of monitoring the terpene content of the living cells before the step of extracting the micelles from the living cells.

15. A method for modifying living cells to cause the living cells to collect a high concentration of terpenes inside the living cells, comprising:
transforming the living cells with an expression vector that causes the living cells to express peptides that are targeted for prenylation, wherein the expression vector comprises a nucleotide sequence that codes for an amino acid sequence of CaaX, wherein C is cysteine, a is any aliphatic amino acid and X is methionine (M), serine (S), glutamine (Q), alanine (A), cysteine (C), leucine (L) or glutamate (E), whereby the living cells express the peptides, the peptides are prenylated with terpene molecules by the living cells to produce prenylated peptides, and the prenylated peptides accumulate into micelles inside the living cells.

16. A modified living cell having a high concentration of terpenes inside the living cell, prepared by the method of claim 15.

17. An expression vector for modifying living cells to facilitate concentration and collection of terpenes inside the living cells, comprising:
a nucleotide sequence that codes for an amino acid sequence of MY(n)TKCaaX, wherein C is cysteine, a is any aliphatic amino acid, X is methionine (M), serine (S), glutamine (Q), alanine (A), cysteine (C), leucine (L) or glutamate (E), T is threonine, and K is lysine, M is methionine, Y is one or more additional amino acids, and n is an integer representing a total number of the amino acids included in Y; and
a nucleotide sequence comprising a promoter specific for the living cells.

18. The expression vector of claim 17, wherein n is 1 and Y is aspartate (D) or glutamate (E).

19. The expression vector of claim 17, wherein n is more than 1 and Y comprises at least one amino acid that is aspartate (D) or glutamate (E) immediately adjacent to the methionine (M).

20. The expression vector of claim 17, further comprising a nucleotide sequence that comprises the Kozak consensus sequence CCATGG.


SEQUENCE LISTING (58 sequences)

SEQ ID NO: 1 - protein, 4 aa, eukaryotic
  X (2)..(2): X is any amino acid
  X (3)..(3): X is any amino acid
  X (4)..(4): X is M, S, Q, A, C, L, or E
CXXX

SEQ ID NO: 2 - protein, 6 aa, eukaryotic
  X (4)..(4): X is any amino acid
  X (5)..(5): X is any amino acid
  X (6)..(6): X is any amino acid
TKCXXX

SEQ ID NO: 3 - protein, 4 aa, eukaryotic
  X (2)..(2): X is any amino acid
  X (3)..(3): X is any amino acid
  X (4)..(4): X is any amino acid
CXXX

SEQ ID NO: 4 - protein, 4 aa, eukaryotic
CAVQ

SEQ ID NO: 5 - protein, 4 aa, eukaryotic
CSIM

SEQ ID NO: 6 - protein, 4 aa, eukaryotic
CLVM

SEQ ID NO: 7 - protein, 4 aa, eukaryotic
CTKF

SEQ ID NO: 8 - protein, 4 aa, eukaryotic
CRIS

SEQ ID NO: 9 - protein, 4 aa, eukaryotic
CCIF

SEQ ID NO: 10 - protein, 4 aa, eukaryotic
CKIS

SEQ ID NO: 11 - protein, 4 aa, eukaryotic
CSIS

SEQ ID NO: 12 - protein, 4 aa, eukaryotic
CYVM

SEQ ID NO: 13 - protein, 4 aa, eukaryotic
CLIT

SEQ ID NO: 14 - protein, 4 aa, eukaryotic
CNIM

SEQ ID NO: 15 - protein, 4 aa, eukaryotic
CTLQ

SEQ ID NO: 16 - protein, 4 aa, eukaryotic
CSVS

SEQ ID NO: 17 - protein, 4 aa, eukaryotic
CSLM

SEQ ID NO: 18 - protein, 4 aa, eukaryotic
CLLM

SEQ ID NO: 19 - protein, 4 aa, eukaryotic
CTVF

SEQ ID NO: 20 - protein, 4 aa, eukaryotic
CSVQ

SEQ ID NO: 21 - protein, 4 aa, eukaryotic
CGIM

SEQ ID NO: 22 - protein, 4 aa, eukaryotic
CTLM

SEQ ID NO: 23 - protein, 4 aa, eukaryotic
CGLY

SEQ ID NO: 24 - protein, 4 aa, eukaryotic
CNLM

SEQ ID NO: 25 - protein, 4 aa, eukaryotic
CYPD

SEQ ID NO: 26 - protein, 4 aa, eukaryotic
CVLQ

SEQ ID NO: 27 - protein, 4 aa, eukaryotic
CITQ

SEQ ID NO: 28 - protein, 4 aa, eukaryotic
CFLS

SEQ ID NO: 29 - protein, 4 aa, eukaryotic
CNLQ

SEQ ID NO: 30 - protein, 4 aa, eukaryotic
CTMS

SEQ ID NO: 31 - protein, 4 aa, eukaryotic
CQTA

SEQ ID NO: 32 - protein, 4 aa, eukaryotic
CVLA

SEQ ID NO: 33 - protein, 4 aa, eukaryotic
CGLF

SEQ ID NO: 34 - protein, 4 aa, eukaryotic
CIVA

SEQ ID NO: 35 - protein, 4 aa, eukaryotic
CSFM

SEQ ID NO: 36 - protein, 4 aa, eukaryotic
CTLA

SEQ ID NO: 37 - protein, 4 aa, eukaryotic
CCIL

SEQ ID NO: 38 - protein, 4 aa, eukaryotic
CVQM

SEQ ID NO: 39 - protein, 4 aa, eukaryotic
CVVT

SEQ ID NO: 40 - protein, 4 aa, eukaryotic
CSLG

SEQ ID NO: 41 - protein, 4 aa, eukaryotic
CSLC

SEQ ID NO: 42 - protein, 4 aa, eukaryotic
CLTS

SEQ ID NO: 43 - protein, 4 aa, eukaryotic
CKIF

SEQ ID NO: 44 - protein, 4 aa, eukaryotic
CNLF

SEQ ID NO: 45 - protein, 4 aa, eukaryotic
CGIA

SEQ ID NO: 46 - protein, 4 aa, eukaryotic
CVLH

SEQ ID NO: 47 - protein, 4 aa, eukaryotic
CLML

SEQ ID NO: 48 - protein, 4 aa, eukaryotic
CTII

SEQ ID NO: 49 - protein, 8 aa, eukaryotic
  X (6)..(6): X is any amino acid
  X (7)..(7): X is any amino acid
  X (8)..(8): X is M, S, Q, A, C, L, or E
MDTKCXXX

SEQ ID NO: 50 - protein, 8 aa, eukaryotic
  X (2)..(2): X is one or more of any amino acid
  X (6)..(6): X is any amino acid
  X (7)..(7): X is any amino acid
  X (8)..(8): X is M, S, Q, A, C, L, or E
MXTKCXXX

SEQ ID NO: 51 - protein, 5 aa, eukaryotic
  X (2)..(2): X is any amino acid
MXTKC

SEQ ID NO: 52 - protein, 13 aa, eukaryotic
  X (11)..(11): X is any amino acid
  X (12)..(12): X is any amino acid
  X (13)..(13): X is M, S, Q, A, C, L, or E
MHHHHHHTKCXXX

SEQ ID NO: 53 - protein, 8 aa, eukaryotic
DYKDDDDK

SEQ ID NO: 54 - protein, 6 aa, eukaryotic
  X (4)..(4): X is any amino acid
  X (5)..(5): X is any amino acid
  X (6)..(6): X is M, S, Q, A, C, L, or E
TKCXXX

SEQ ID NO: 55 - protein, 8 aa, eukaryotic
  X (2)..(2): X is one or two of D or E, or D-X or E-X
  X (6)..(6): X is any amino acid
  X (7)..(7): X is any amino acid
  X (8)..(8): X is M, S, Q, A, C, L, or E
MXTKCXXX

SEQ ID NO: 56 - DNA, 32 bp, Artificial Sequence
  coding sequence of peptide targeted for prenylation
ccatggacaccaagtgcgccgtgcagtgatga

SEQ ID NO: 57 - DNA, 32 bp, Artificial Sequence
  complementary coding sequence for peptide targeted for prenylation
ggtacctgtggttcacgcggcacgtcactact

SEQ ID NO: 58 - protein, 8 aa, Artificial Sequence
  sequence for peptide targeted for prenylation
MDTKCAVQ